(19) United States
(12) Patent Application Publication
(10) Pub. No.: US 2026/0147129 A1
(43) Pub. Date: May 28, 2026

(54) SENSOR SYSTEMS FOR MEDICAL DEVICES, MOBILE IMAGING VEHICLES, AND WORK VEHICLES

(71) Applicant: GE Precision Healthcare LLC, Waukesha, WI (US)

(72) Inventors: Jimmie Beacham, West Ellis, WI (US); Gerald McKenzie, Florence, SC (US); Megan Stoychoff, East Troy, WI (US); Tricia Clark, Oconomowoc, WI (US)

(73) Assignee: GE Precision Healthcare LLC, Waukesha, WI (US)

(21) Appl. No.: 18/917,884

(22) Filed: Oct. 16, 2024

(51) Int. Cl. G01V 1/18; A61B 5/055; B60K 35/28; G01H 1/00

(52) U.S. Cl. CPC G01V 1/18; A61B 5/055; B60K 35/28; G01H 1/00

(57) ABSTRACT

A system includes a vibration sensor configured to sense vibrations and to generate vibration data based on the sensed vibrations, a medical imaging device coupled with the vibration sensor and comprising a display device, the medical imaging device being configured to obtain medical images of a patient, and a processing circuit configured to receive the vibration data from the vibration sensor, and provide an output on the display device based on the vibration data.

### FIELD

Embodiments of the subject matter disclosed herein relate to sensing vibrations in conjunction with medical imaging, and sensing ground voids in relation to work vehicles, and more particularly, providing operators of such systems outputs in relation to sensed vibrations or ground voids that may affect operation of the same.

### BACKGROUND

Unexpected vibrations may affect the integrity of medical images obtained by medical imaging equipment, such as magnetic resonance (MR) imaging devices, computed tomography (CT) imaging devices, positron emission tomography (PET) imaging devices, single-photon emission computed tomography (SPECT) imaging devices, or X-ray imaging devices. The vibrations may be caused by an external source, such as from seismic activity, other people, or from the operation of other equipment. The vibrations may also be caused by the medical imaging equipment itself, such as from a worn or misaligned part. Ground voids may also cause work vehicles to become unsteady when lifting heavy equipment, such as the magnets of MR imaging devices.

### SUMMARY

An embodiment relates to a system. The system includes a vibration sensor, a medical imaging device, and a processing circuit. The vibration sensor is configured to sense vibrations and to generate vibration data based on the sensed vibrations. The medical imaging device is coupled with the vibration sensor and includes a display device. The medical imaging device is configured to obtain medical images of a patient. The processing circuit is configured to receive the vibration data from the vibration sensor and provide an output on the display device based on the vibration data.

Another embodiment relates to a system. The system includes a ground sensor system, a work vehicle, and a processing circuit. The ground sensor system is configured to sense at least one of seismic activity or underground voids, and to generate ground sensor data based on the sensed seismic activity. The work vehicle includes the seismic sensor and a display device. The work vehicle is configured to lift equipment. The processing circuit on the work vehicle and is configured to receive the ground sensor data from the ground sensor system and provide an output on the display device based on the ground sensor data.

Another embodiment relates to a method. The method includes generating, by a vibration sensor configured to generate vibration data based on sensed vibrations and/or vibration data. The method further includes receiving, by a processing circuit of a medical imaging device configured to obtain medical images of a patient and comprising the vibration sensor and a display device, the vibration data from the vibration sensor. The method further includes providing, by the processing circuit via the display device, an output based on the vibration data.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

FIG. 1 is a block diagram of a sensor system configured to sense vibrations and ground voids, according to an example embodiment.

FIG. 2 is a block diagram of a medical imaging system including the sensor system of FIG. 1, according to an example embodiment.

FIG. 3 is an illustration of a mobile medical imaging vehicle including the medical imaging system of FIG. 2 including the sensor system of FIG. 1, according to an example embodiment.

FIG. 4 is a flow chart of a method of operating the sensor system of the mobile medical imaging vehicle of FIG. 3, according to an example embodiment.

FIG. 5 is a flow chart of a method of operating the sensor system of the medical imaging system of FIG. 2 or the mobile medical imaging vehicle of FIG. 3 prior to performing a medical imaging process, according to an example embodiment.

FIG. 6 is a flow chart of a method of operating the sensor system of the medical imaging system of FIG. 2 or the mobile medical imaging vehicle of FIG. 3 during a medical imaging process, according to an example embodiment.

FIG. 7 is an illustration of a work vehicle comprising the sensor system of FIG. 1, according to an example embodiment.

FIG. 8A is an illustration of outrigging feet comprising a sensor of the sensor system of FIG. 1, according to an example embodiment.

FIG. 8B is another illustration of outrigging feet comprising a sensor of the sensor system of FIG. 1, according to an example embodiment.

FIG. 9 is a flow chart of a method of operating the sensor system of the work vehicle of FIG. 7, according to an example embodiment.

FIG. 10 is a flow chart of another method of operating the work vehicle of FIG. 7, according to an example embodiment.

### DETAILED DESCRIPTION

Referring generally to the figures, sensor systems for medical devices, mobile imaging vehicles, or work vehicles are disclosed. The sensor system can be integrated with a medical imaging device, such as a magnetic resonance (MR) imaging system, among others, to determine whether detected vibrations are likely to affect the image quality of images taken during a medical imaging process or indicate the existence of a maintenance issue. The sensor system can also be integrated with a mobile medical imaging vehicle to similarly alert the operator of external vibrations that may affect imaging, providing the operator with an instruction to relocate the mobile medical imaging vehicle. The sensor system can likewise be integrated with a work vehicle, such as a crane for lifting heavy objects like components of magnetic resonance imaging systems, to detect underground voids that may cause the work vehicle to become unstable during operation.

External vibrations can significantly distort images obtained using medical imaging equipment such as MR imaging systems, primarily because MR imaging systems rely on highly sensitive magnetic fields and radiofrequency signals to capture detailed images of internal body structures. The MR imaging process requires the patient and the imaging equipment to remain still while the machine generates magnetic fields and detects tiny shifts in hydrogen atoms within the body. Any external vibrations—whether from nearby machinery, foot traffic, building infrastructure, or geological events—can cause subtle shifts in the position of the patient or the machine's components, leading to image blurring or misalignment of the scanned slices. Even small vibrations can introduce enough noise into the system to affect the clarity and accuracy of the images. For example, even slight vibrations can cause the gradient coil of an MR imaging system to become misaligned, leading to spatial inaccuracies in the imaging data, resulting in geometric distortions or blurred edges in the final images. For instance, if an MR imaging scan is being used to detect a small tumor or lesion, external vibrations could cause the image to appear fuzzy, making it difficult for radiologists to interpret the scan accurately or lead to misdiagnosis.

External vibrations can also impact the magnet stability of the MR imaging system. MR imaging systems often have superconducting magnets that must remain stable to maintain a consistent magnetic field. Vibrations from sources like HVAC systems, elevators, or nearby construction can interfere with this stability, causing fluctuations in the magnetic field during the scan. These fluctuations introduce artifacts or noise into the images, which can appear as streaks, ghosting, or random distortions.

Operating medical imaging equipment, such as an MR imaging system or a computed tomography (CT) imaging system, that are in need of maintenance can also cause premature wear of the device or deficient imaging outcomes. For example, operating a CT imaging system with a worn bearing or an off-balance gantry can severely impact both the machine's functionality and the quality of the diagnostic images. For example, a worn bearing may cause excessive friction and vibration during the rapid rotation of the gantry, leading to mechanical instability, which can produce blurry or distorted images, thereby compromising diagnostic accuracy. Additionally, the imbalance can place undue stress on other mechanical components, accelerating wear and potentially leading to sudden equipment failure. Operating medical imaging equipment in such a compromised state risks costly repairs and poses safety hazards to both patients and technicians.

Furthermore, operating a work vehicle employing outriggers for stability, such as a crane, can lead to catastrophic instability if the outriggers are inadvertently deployed over underground voids such as manhole covers, sewer lines, caves, or sinkholes. When the outriggers are deployed, and particularly when the outriggers are deployed and the work vehicle lifts a heavy object such as a magnet for an MR imaging system, the outriggers exert significant pressure on the ground. If the ground beneath an outrigger contains an undetected underground void, the weight of the work vehicle can cause the ground to collapse. This sudden loss of support may cause the outrigger to sink or shift unexpectedly, leading to the crane tipping over, becoming severely unbalanced, or dropping its load. Such an event not only risks damaging the crane and the load but also endangers the safety of the operators and nearby workers. To mitigate this risk, it is advantageous to scan the area, and particularly the area beneath each outrigger, for underground voids before fully deploying the outriggers and lifting a load.

Using a vibration sensor in conjunction with medical imaging equipment such as an MR imaging system or a CT imaging system provides a significant technological improvement by actively monitoring and mitigating the effects of external vibrations that could distort images or identifying maintenance issues before the maintenance issue distorts images. By detecting even subtle vibrations in real time, the vibration sensor can alert the system operator to pause the scan, relocate the imaging equipment to another location in the case of a mobile medical imaging vehicle, or alert maintenance personal to a maintenance issue. Such detection and notification ensures that high-quality medical images are captured without interference. This prevents the need for rescans due to image blurring, saving time and reducing the patient's exposure to the radiation of the scanning equipment. Moreover, by addressing vibration issues before performing a medical imaging process, the system avoids relying on extensive post-processing corrections to fix image distortions, which saves processing power and computer resources. The integration of vibration sensors thus enhances the precision and efficiency of medical imaging technology, optimizing both the diagnostic process and the operational performance of the imaging equipment.

Using a ground sensor in conjunction with a work vehicle such as a crane provides a technological improvement that enhances safety and efficiency by detecting underground voids or unstable soil conditions before fully deploying outriggers. The sensors enable the operator of the work vehicle to assess the ground's integrity in real time, ensuring that the outriggers are positioned on solid, stable ground. This proactive detection reduces the risk of accidents, or in the case of a crane, instability during heavy lifts, such as when lifting a magnet for installation in an MR imaging system. By preventing the potential collapse of outriggers into hidden voids, the system eliminates the need for manual ground inspections or corrective actions after a failure occurs. Additionally, this system saves processing power by reducing the reliance on complex structural calculations to compensate for an unstable work vehicle setup, allowing for smoother and more reliable operations of the work vehicle.

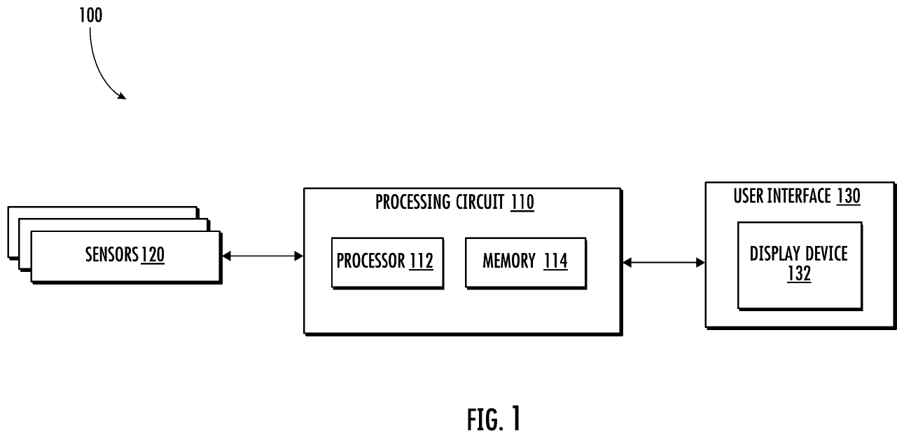

Referring to FIG. 1, a block diagram of a sensor system 100 configured to sense vibrations and ground voids is shown according to an example embodiment. The sensor system 100 includes a processing circuit 110, a plurality of sensors 120, and a user interface 130. As will be described in greater detail below, the sensor system 100 may be part of medical imaging equipment used in a medical environment (e.g., hospitals, clinics, mobile medical imaging, etc.), or part of a work vehicle in a work environment (e.g., construction site, engineering project site).

The processing circuit 110 includes a processor 112 and memory 114 which are configured to carry out the functions of the sensor system 100. The processor 112 may include a CPU, a GPU, a microprocessor, a DSP, a general-purpose single-or multi-chip processor, a field-programmable gate array (FPGA), or any other type of processor capable of performing logical operations. A general-purpose processor may be a microprocessor, or, any conventional processor, or state machine. A processor also may be implemented as a combination of computing devices, such as a combination of a DSP and a microprocessor, a plurality of microprocessors, one or more microprocessors in conjunction with a DSP core, or any other such configuration. In some embodiments, the processor 112 may be shared by multiple circuits. Alternatively or additionally, the processor 112 may be structured to perform or otherwise execute certain operations independent of one or more co-processors. In some embodiments, two or more processors may be coupled via a bus to enable independent, parallel, pipelined, or multi-threaded instruction execution. All such variations are intended to fall within the scope of the present disclosure.

In some embodiments, the processing circuit 110 may include multiple processors configured to perform the processing operations/functionality described with reference to processor 112. It should be appreciated that other embodiments may use a different arrangement of processors. The processor 112 may be in electronic communication with the user interface 130 and the display device 132 such that the processor 112 may process data obtained by the sensors 120 and generate images or other information to display on the display device 132.

The memory 114 may be configured to, for example, store processed or unprocessed volumes of data obtained by the sensor system 100 (e.g., vibration data, seismic data, or ground void data collected by the sensors 120). For example, the memory 114 may be a hospital picture archiving and communication system (PACS). The memory 114 (e.g., memory, memory unit, storage device, etc.) may include one or more devices (e.g., RAM, ROM, Flash memory, hard disk storage, etc.) for storing data and/or computer code for completing or facilitating the processes, layers, and modules described in the present application. The memory 114 may be or include tangible, non-transient volatile memory or non-volatile memory. The memory 114 may also include database components, object code components, script components, or any other type of information structure for supporting the activities and information structures described in the present application.

In various embodiments, the memory 114 may have varying capacity (e.g., storage space) across embodiments of the sensor system 100. For example, the memory 114 may be configured to store sensor data obtained over several days or years of operation of the sensor system 100. The sensor data may be stored in the memory 114 such that the sensor data may be retrieved according to an order/time of acquiring the data. That is, the sensor data may be stored with a timestamp indicating a time at which the sensor data was collected and may be retrieved starting with an oldest time at which the sensor data was collected.

The sensors 120 can include one or more of any type of sensor configured to sense vibrations, seismic activity, or ground voids. In some embodiments, the sensors 120 include a vibration sensor. In some embodiments, the sensors 120 are configured to sense vibrations and to generate vibration data based on the sensed vibrations. In some embodiments, the sensor 120 is configured to detect mechanical movement caused by vibrations in its environment. The mechanical movement is then converted into an electrical signal through a transducer of the sensor 120, which in some examples can be a piezoelectric element, a coil, or an accelerometer mass. For example, when an object in a sensing range of the vibration sensor vibrates, the vibration sensor can detect the movement through an internal mechanism, such as through a mass inside an accelerometer or a deformation in a piezoelectric material. In some embodiments, as the object moves, the internal components of the sensor are displaced, and this mechanical movement is converted into a proportional electrical signal, such as a voltage or current for output to the processing circuit 110. In some embodiments, the amplitude of the vibration corresponds to the strength of the signal, such that a larger vibration will produce a higher voltage indicating a relatively stronger or more intense movement. In some embodiments, the vibration sensor also detects the frequency of the vibration based on how often the movements occur, and converts the frequency into corresponding electrical pulses for output to the processing circuit 110.

In some embodiments, the sensor 120 is configured to preprocess the electrical signal before providing an output to the processing circuit 110, such as by conditioning the electrical signal by amplifying, filtering, or digitizing the electrical signal to clarify the signal and reduce noise. The sensor 120 is configured to provide the preprocessed signal to the processing circuit 110 as vibration data where it can be further analyzed or processed.

In some embodiments, the vibration sensor is configured to generate vibration data prior to a medical imaging device obtaining a medical image of a patient. In some embodiments, the vibration sensor is configured to generate vibration data during a medical imaging process. In some embodiments, the vibration data is indicative of a component of the medical imaging device requiring maintenance. For example, the vibration sensor may be a seismometer, a geophone, an accelerometer, or an infrasound sensor, among other sensor types. While any type of vibration sensor can be used by the medical imaging system 200 or the mobile medical imaging vehicle 300, a particular vibration sensor may be chosen that is best suited for a particular use case.

In some embodiments, the sensors 120 include a ground sensor configured to sense at least one of seismic activity or underground voids, and to generate ground sensor data based on the sensed seismic activity. In this example, the ground sensor may be a ground-penetrating radar sensor, a microgravity sensor, or a seismic sensor such as a vibroseis device or a hammer and plate device. A ground penetrating radar sensor is a non-invasive method that uses high-frequency electromagnetic waves to detect underground structures such as voids. A ground penetrating radar sensor can include an antenna that transmits radar pulses into the ground and a receiver that captures the reflected signals. A microgravity sensor can detect underground voids by measuring minute variations in the Earth's gravitational field. For example, a microgravity sensor can use a gravimeter to detect the presence of a void (e.g., an air-or water-filled cavity) by detecting a slight decrease in the local gravitational pull. A seismic sensor can detect underground voids by analyzing how waves travel through earth. In some embodiments, the seismic sensor can generate seismic waves and detect the speed and characteristics of the waves as they interact with underground structures. For example, the seismic sensor can generate seismic waves using a vibroseis device that includes a large vibrating plate that is coupled to the ground and vibrated. In another example, the seismic sensor can generate seismic waves using a hammer and plate device that includes a hammer that strikes a plate coupled to the ground. The seismic sensor can include a network of receivers, such as geophones, that receive the waves as they return to the surface after interacting with the ground structures. The seismic sensor can detect underground voids by analyzing the received waves for variations that would be indicative of underground voids (e.g., waves slowing down or exhibiting unusual reflections).

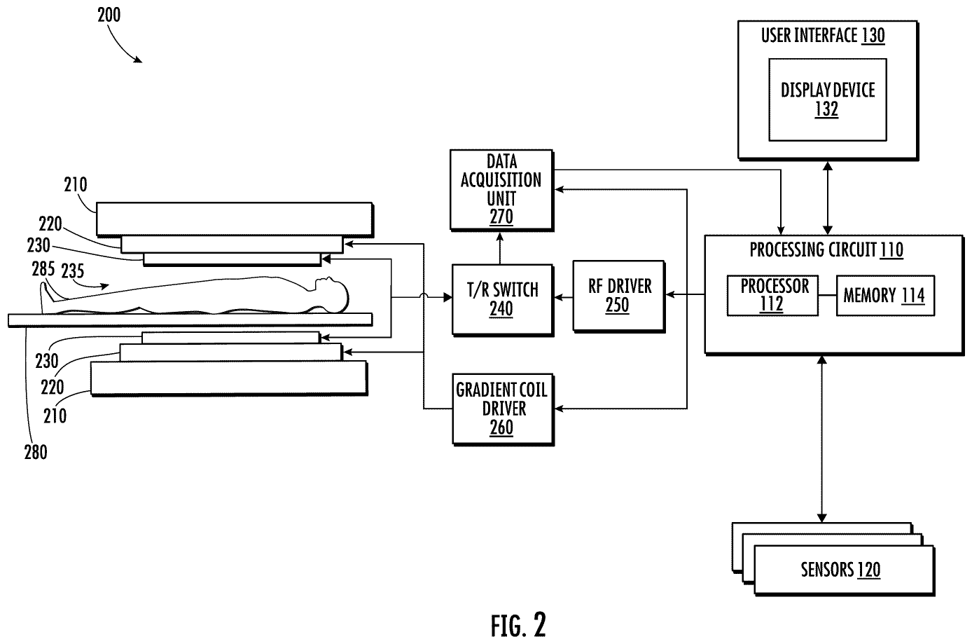

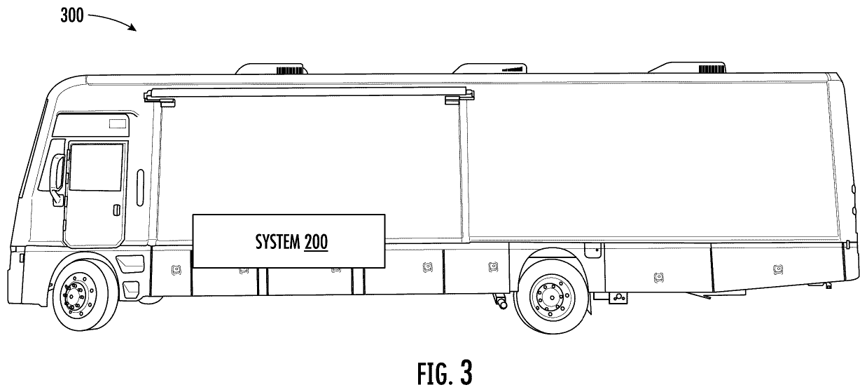

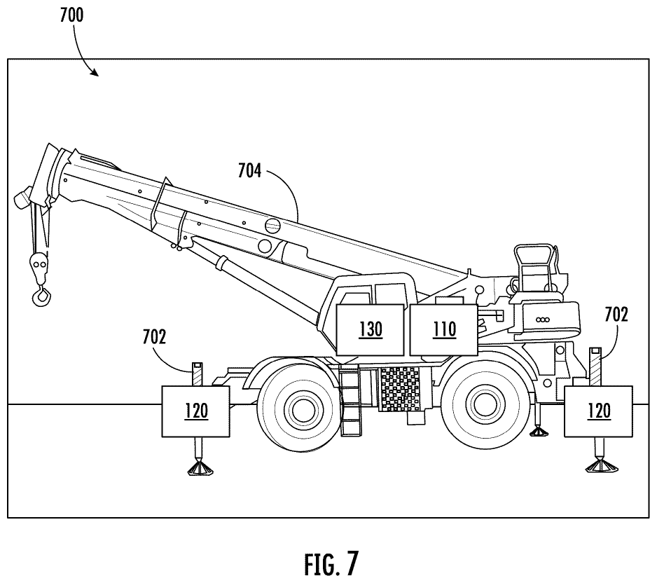

The user interface 130 may be used by an operator of a medical imaging system (e.g., a technician or clinician), such as the medical imaging system 200 described with reference to FIG. 2, or an operator of a vehicle (e.g., driver or vehicle technician), such as the mobile medical imaging vehicle 300 described with reference to FIG. 3, including sensor system 100 to control operation of the medical imaging system or vehicle, respectively. The user interface 130 may also be used by an operator of the work vehicle 700 described with reference to FIG. 7. For example, an operator of the medical imaging system 200 may use the user interface 130 to control the input of patient data, to change a scanning or display parameter, and/or to select various other modes, operations, parameters, etc. of the sensor system 100. In some embodiments, the user interface 130 may include an off-the-shelf consumer electronic device such as a smartphone, a tablet, a laptop, and so on. For the purposes of this disclosure, the term “off-the-shelf consumer electronic device” is defined to be an electronic device that was designed and developed for general consumer use and one that was not specifically designed for use in a medical environment. Alternatively, in other embodiments, the user interface 130 may be an electronic device that was designed and developed for use in a medical environment or vehicle environment.

According to some embodiments, the user interface 130 may be physically separate from the rest of the sensor system 100. The user interface 130 may communicate with the processor 112 through a wireless protocol, such as Wi-Fi, Bluetooth, wireless local area network (WLAN), near-field communication, and so on. According to some embodiments, the user interface 130 may communicate with the processor 112 through an application programming interface (API).

In some embodiments, the user interface 130 may include physical controls such as one or more of buttons, sliders, a rotary knob, a mouse, a keyboard, a trackball, a steering wheel, hard keys linked to specific actions, soft keys that may be configured to control different functions, and so on. In some embodiments, the user interface includes a speaker or a microphone. As shown in FIG. 1, the user interface 130 may also include a display device 132. In some embodiments, the display device 132 may be configured to display a graphical user interface (GUI) based on an instruction from the memory 114. The GUI may include user interface icons representing commands and instructions relating to the operation of the sensor system 100. The user interface icons of the GUI may be configured such that a user may select a specific user interface icon in order to initiate a specific function controlled by the GUI. For example, various user interface icons may be used to represent windows, menus, buttons, cursors, scroll bars, and so on. That is, the physical controls of the user interface 130 may be included as individual hardware elements, as user interface icons displayed on the display device 132, or as a combination of hardware elements and user interface icons.

In some embodiments, the display device 132 may include a touch-sensitive display device or a touch screen. According to such embodiments, the touch screen may be configured to interact with the GUI displayed by the display device 132 such that a user can interact with the GUI via the touch screen. The touch screen may be a single-point touch screen that is configured to detect a single contact point at a time, or the touch screen may be a multi-point touch screen that is configured to detect multiple points of contact at a time. For embodiments where the touch screen is a multi-point touch screen, the touch screen may be configured to detect multi-point gestures involving contact from two or more of a user's fingers at a time. The touch screen may be a resistive touch screen, a capacitive touch screen, or any other type of touch screen that is configured to receive inputs from a stylus or one or more of a user's fingers. According to some embodiments, the touch screen may be an optical touch screen that uses technology such as infrared light or other frequencies of light to detect one or more points of contact initiated by a user. In some embodiments, the touch screen may be incorporated as part of the display device 132 or may be separate from the display device 132. The user interface 130 may also include a proximity sensor configured to detect objects and/or gestures that are within a predetermine distance (e.g., five feet, six inches, ten centimeters, etc.) of the proximity sensor. In various embodiments, the proximity sensor may be located on the display device 132 or as part of a touch screen that is separate from the display device 132.

The processing circuit 110 may be configured to receive data from the sensors 120, analyze the data, make determinations based on the data, or otherwise process the data, and to provide an output or control signal based on the same. In some embodiments, the processing circuit 110 is configured to receive vibration data from a vibration sensor, or ground sensor data from a ground sensor, and provide an output on the display device 132 based on the vibration data or ground sensor data, respectively.

In some embodiments, the processing circuit 110 is configured to provide the output prior to the medical imaging system 200 obtaining a medical image of the patient 285. In some embodiments, the output provides an indication of whether any vibrations were sensed and whether the medical imaging system 200 can be operated to obtain a medical image without comprising the integrity of the medical image. In some embodiments, the processing circuit 110 is configured to provide the output at least one of during a medical imaging process or after the medical imaging process. In some embodiments, the output comprises an indication of whether any vibrations were sensed during the medical imaging process and whether the integrity of any obtained medical images is compromised. In some embodiments, the output identifies a specific medical image of a plurality of the obtained medical images that is comprised based on the vibration data. In some embodiments, the output causes the medical imaging system 200 to stop the medical imaging process based on the vibration data. Additional capabilities of the processing circuit 110 will be explained further herein.

Referring to FIG. 2, a block diagram of a medical imaging system 200 including the sensor system 100 of FIG. 1 is shown according to an example embodiment. In some embodiments, as shown in FIG. 2, the sensor system 100 is integrated with a medical imaging system, such as the medical imaging system 200. The medical imaging system 200 is configured to obtain medical images of a patient. As shown in FIG. 2, the medical imaging system 200 is a magnetic resonance (MR) imaging system. However, it will be appreciated that the medical imaging system 200 can be any type of medical imaging system. For example, the medical imaging system 200 can be or include at least one of an MR imaging device, a computed tomography (CT) imaging device, a positron emission tomography (PET) imaging device, a single-photon emission computed tomography (SPECT) imaging device, or an X-ray imaging device, among others.

The medical imaging system 200 includes a magnetostatic field magnet 210, a gradient coil 220, an RF body coil 230, a transmit/receive (T/R) switch 240, an RF driver 250, a gradient coil driver 260, a data acquisition unit 270, a patient bed 280 for a patient 285, a processing circuit 110, a plurality of sensors 120, and a user interface 130. It will be appreciated that the processing circuit 110, the plurality of sensors 120, and the user interface 130 is shown to be integrated with the medical imaging system 200. However, it will be appreciated that the processing circuit 110, the plurality of sensors 120, and the user interface 130 can be separate from the medical imaging system 200, and the medical imaging system 200 may instead include its own dedicated processing circuit and user interface.

The medical imaging system 200 is configured to transmit electromagnetic pulse signals to the patient 285 when the patient 285 is on the patient bed 280 within an imaging space 235 having a magnetostatic field formed therein to scan the patient 285 as part of a medical imaging process. The medical imaging system 200 is operable to obtain magnetic resonance (MR) signals from the patient 285 to construct an image of a slice of the patient 285 from a series of images based on the MR signals obtained through the medical imaging process.

The magnetostatic field magnet 210 includes an annular superconducting magnet mounted within a toroidal vacuum vessel. The magnet defines a cylindrical space surrounding the patient 285, and generates a constant primary magnetostatic field.

The gradient coil 220 generates a gradient magnetic field within the imaging space 235, providing three-dimensional positional information for the magnetic resonance signals received by RF coil arrays (not shown). The gradient coil 220 includes three gradient coil systems, each producing a gradient magnetic field along one of the three mutually perpendicular spatial axes. These fields are applied in the frequency encoding direction, phase encoding direction, and slice selection direction based on the imaging requirements. Specifically, the gradient coil 220 creates a gradient field along the slice selection (or scan) direction to select the desired slice of the patient 285, and the RF body coil 230 or local RF coil arrays transmit an RF pulse to that slice. The gradient coil 220 also generates a gradient field in the phase encoding direction of the patient 285 to phase encode the magnetic resonance signals from the excited slice, and then applies a gradient field in the frequency encoding direction of the patient 285 to frequency encode the magnetic resonance signals from that slice.

The RF coil arrays enclose the region to be imaged of the patient 285. The RF coil arrays can transmit an RF pulse comprising an electromagnet waive to the patient 285 to generate a high frequency magnetic field. The RF coil arrays transmit the RF pulse in the imaging space 235 based on a signal received from the processing circuit 110. The high frequency magnetic field excites a spin of protons in the slice to be imaged of the patient 285. The RF coil arrays receive, as a MR signal, the electromagnetic wave generated when the proton spin returns into alignment with an initial magnetization vector. In some embodiments, each RF coil can transmit and receive an RF pulse using the same RF coil. In some embodiments, the RF coil may only receive MR signals, and not transmit the RF pulse. Different RF coil arrays may be utilized for different scanning objectives. Accordingly, one or more of the RF coil arrays may be disconnected from the medical imaging system 200 so that a different coil array may be connected to the medical imaging system 200. The RF coil arrays may be coupled to the RF driver 250 and the data acquisition unit 270 via the T/R switch 240.

The RF body coil 230 encloses the imaging space 235 and generates RF magnetic field pulses orthogonal to a main magnetic field produced by the magnetostatic field magnet 210 within the imaging space 235 to excite the nuclei of the patient 285. The RF body coil 230 is fixed to the medical imaging system 200. In some embodiments, the RF body coil 230 has a larger coverage area than the RF coil arrays and can be used to transmit or receive signals to the whole body of the patient 285. In some embodiments, using receive-only RF coil arrays and transmit body coils, the medical imaging system 200 can provide a uniform RF excitation and good image uniformity at the expense of high RF power provided to the patient 285. In some embodiments, using a transmit-receive RF coil array, the medical imaging system 200 provides the RF excitation to the region of interest and receives the MR signal, thereby decreasing the RF power provided to the patient 285. The RF body coil 230 can be configured to operate in a transmit-only mode, a receive-only mode, or a transmit-receive mode. The RF coil arrays can be configured to operate in a transmit-receive mode or a receive-only mode.

The T/R switch 240 is configured to selectively connect the RF body coil 230 to the data acquisition unit 270 when operating in a receive mode, and to the RF driver 250 when operating in a transmit mode. The T/R switch 240 can selectively electrically connect one or more of the RF coil arrays to the data acquisition unit 270 when the RF coil arrays operate in the receive mode, and to the RF driver 250 when RF coil arrays operate in the transmit mode. In some embodiments, when the RF coil arrays and the RF body coil 230 are both used in a single scan and the RF coil arrays are configured to receive MR signals and the RF body coil 230 is configured to transmit RF signals, the T/R switch 240 is configured to direct control signals from the RF driver 250 to the RF body coil 230 and to direct received MR signals from the RF coil arrays to the data acquisition unit 270.

The RF driver 250 includes a gate modulator, an RF power amplifier, and an RF oscillator configured to drive the RF coil arrays and create a high-frequency magnetic field in the imaging space 235. The RF driver 250 is configured to modulate, based on a control signal from the processing circuit 110 and using the gate modulator, the RF signal received from the RF oscillator into a signal of predetermined timing having a predetermined envelope. The RF power amplifier is configured to amplify the RF signal modulated by the gate modulator and then the modulated RF signal is output to the RF coil arrays.

The gradient coil driver 260 is configured to drive the gradient coil 220 based on a control signal received from the processing circuit 110 and thereby generate a gradient magnetic field in the imaging space 235. The gradient coil driver 260 includes three driver circuits that correspond to each of the three gradient coil systems of the gradient coil 220.

The data acquisition unit 270 includes a preamplifier, a phase detector, and an analog/digital converter configured to acquire the MR signals received by the RF coil arrays. The phase detector is configured to phase detect, using an output from the RF oscillator of the RF driver 250 as a reference signal, the MR signals received from the RF coil arrays and amplified by the preamplifier. The phase detector is configured to output a phase-detected analog magnetic resonance signals to the analog/digital converter for conversion into digital signals. The digital signals are then output to the processing circuit 110.

The patient bed 280 is a table or other surface configured to support the patient 285. The patient can be placed on the patient bed 280 and then moved into the imaging space 235 of the medical imaging device 200. The patient bed 280 may selectively move, with the patient 285 disposed on the patient bed 280, into and out of the imaging space 235 based on control signals received from the processing circuit 110. In some embodiments, one or more RF coil arrays are coupled to the patient bed 280 and move with the patient 285.

The processing circuit 110 is configured to control the operations of the medical imaging device 200 based on user inputs received via the user interface 130. For example, based on a user input, the processing circuit 110 can control the various parts of the medical imaging device 200 (e.g., the patient bed 280, the RF driver 250, gradient coil driver 260, and data acquisition unit 270) to carry out operations to perform a predetermined medical imaging process on the patient 285. The processing circuit 110 can carry out all processes described herein with respect to processing circuit 110.

The plurality of sensors 120 can include any type of sensor configured to detect vibrations. As such, the sensors 120 can be a vibration sensor as described above with respect to FIG. 1.

The user interface 130 can include any type of control elements configured to enable an operator or technician of the medical imaging device 200 to interact with the medical imaging device 200 and to enter commands to control the medical imaging device 200. The user interface 130 can include any feature or functionality described above with reference to FIG. 1.

Referring to FIG. 3, an illustration of a mobile medical imaging vehicle 300 including the medical imaging system 200 of FIG. 2 is shown according to an example embodiment.

The mobile medical imaging vehicle 300 can be any type of vehicle that can house or operate a medical imaging device, such as the medical imaging system 200 described with reference to FIG. 2. However, it will be appreciated that the mobile medical imaging vehicle 300 can include any type of medical imaging device, such as at least one of a magnetic resonance (MR) imaging device, a computed tomography (CT) imaging device, a positron emission tomography (PET) imaging device, a single-photon emission computed tomography (SPECT) imaging device, or an X-ray imaging device.

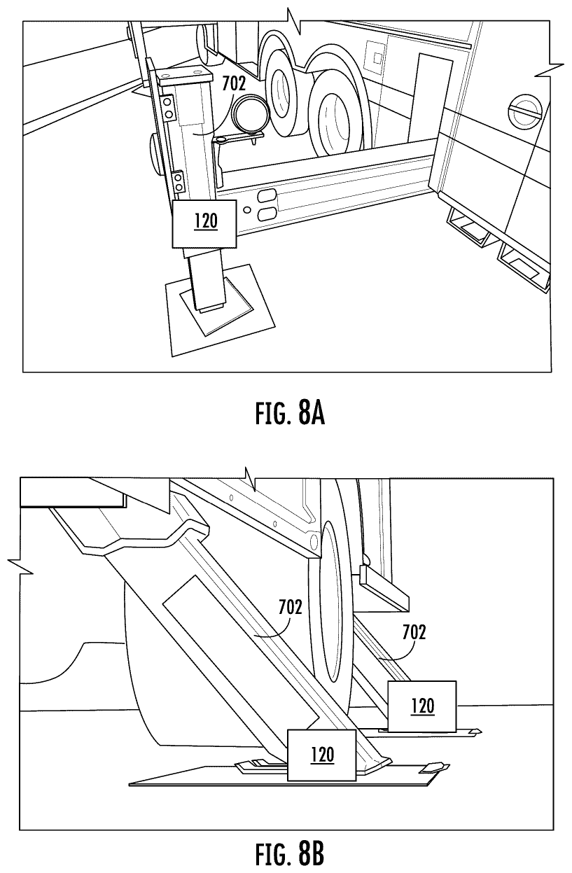

The mobile medical imaging vehicle 300 can be a fully functional diagnostic unit capable of traveling to different locations for purposes of performing medical imaging. Accordingly, the mobile medical imaging vehicle 300 can be a bus, a van, a truck, or a trailer. In some embodiments, the mobile medical imaging vehicle 300 is custom-built on a heavy-duty commercial chassis to support the weight of the medical imaging equipment and additional onboard systems. The mobile medical imaging vehicle 300 can be equipped with a reinforced suspension and a stable platform to minimize vibrations while driving to ensure the integrity of sensitive medical equipment onboard. The mobile medical imaging vehicle 300 can be equipped with specialized compartments to store power generators, ventilation systems, and other utilities needed for the operation of the equipment onboard. In some embodiments, the mobile medical imaging vehicle 300 is equipped with one or more outriggers 702, described in more detail with reference to FIG. 7, FIG. 8A, and FIG. 8B.

The mobile medical imaging vehicle 300 can include an imaging suite inside of the vehicle. The imaging suite can house the onboard medical imaging devices and other medical equipment. In some embodiments, the devices and equipment are installed with vibration-dampening mounts and reinforced structures to ensure they remain calibrated and stable during travel. The mobile medical imaging vehicle 300 can include radiation shielding, such as shielding in the form of lead-lined walls and doors, to ensure operators and patients are shielded from excessive radiation exposure.

The mobile medical imaging vehicle 300 also includes support systems, such as computer systems for operating the medical imaging devices onboard and for keeping patient records. The support systems may be located in a separate room as the imaging suite. The mobile medical imaging vehicle 300 can also include other features such as a waiting area, an examination table, or a changing room.

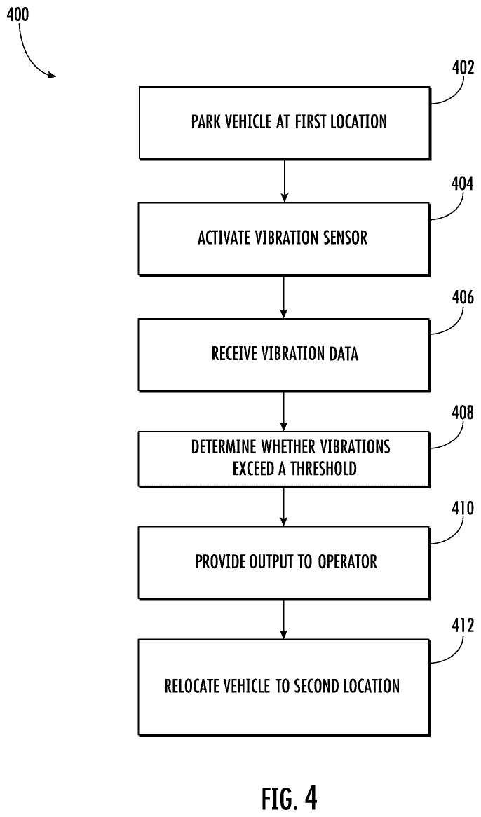

Referring to FIG. 4, a flow chart of a method 400 of operating a sensor system of a mobile medical imaging vehicle is shown according to an example embodiment. In some embodiments, the sensor system referred to by method 400 is the sensor system 100 described above with reference to FIG. 1, the mobile medical imaging vehicle referred to by method 400 is the mobile medical imaging vehicle 300 described above with reference to FIG. 3, including the medical imaging system 200 described above with reference to FIG. 2. In some embodiments, one or more steps of method 400 may be implemented as executable instructions stored in memory onboard the mobile medical imaging vehicle 300, such as memory 114.

At step 402, the mobile medical imaging vehicle 300 is parked at a first location. Typically, an operator of the mobile medical imaging vehicle 300 will drive the mobile medical imaging vehicle 300 to a location in which the medical imaging process described above with reference to the medical imaging system 200 will be carried out. In some embodiments, if the mobile medical imaging vehicle 300 has self-driving capabilities, the mobile medical imaging vehicle 300 can drive to and park at the location without the assistance of a human operator, or with some assistance from the human operator depending on the level of the self-driving capabilities of the mobile medical imaging vehicle 300. In some embodiments, the mobile medical imaging vehicle 300 is already located at a medical imaging location, such as the first location. For example, the mobile medical imaging vehicle 300 may be located at a medical imaging location for a long-term time period (e.g., a week, a month, six months, a year or more).

At step 404, a vibration sensor onboard the mobile medical imaging vehicle 300 is activated. The vibration sensor may be the same as the vibration sensor 120 described above with reference to the sensor system 100 described in FIG. 1. In some embodiments, the vibration sensor 120 is or includes a seismometer, a geophone, an accelerometer, or an infrasound sensor, among other sensor types.

In some embodiments, when a sensor 120 of the sensor system 100 is activated, the sensor 120 receives vibration data. In other words, the sensor 120 detects mechanical movement caused by vibrations in its environment. The mechanical movement is then converted into an electrical signal through a transducer of the sensor 120, which in some examples can be a piezoelectric element, a coil, or an accelerometer mass. In some embodiments, the sensor 120 is configured to preprocess the electrical signal before providing an output to the processing circuit 110, such as by conditioning the electrical signal by amplifying, filtering, or digitizing the electrical signal to clarify the signal and reduce noise. The sensor 120 is configured to provide the preprocessed signal to the processing circuit 110 as vibration data where it can be further analyzed or processed.

At step 406, the processing circuit 110 of the sensor system 100 onboard the mobile medical imaging vehicle 300 receives vibration data.

At step 408, the processing circuit 110 of the sensor system 100 onboard the mobile medical imaging vehicle 300 determines whether the received vibration data is indicative of vibrations exceeding a threshold. In determining whether the received vibration data is indicative of vibrations exceeding a threshold, the processing circuit 110 can compare one or more characteristics of the vibration data to a plurality of thresholds. For example, the thresholds can include at least one of an amplitude threshold defining a maximum allowable vibration level or a frequency threshold defining one or more frequencies or frequency ranges. In some embodiments, the one or more thresholds can include a duration or a pattern. For example, in some embodiments, short-term transient vibrations may not pose a significant risk to obtaining certain types of medical images but prolonged or repetitive vibrations may pose a greater risk to medical imaging equipment, and therefore prolonged or repetitive vibrations may exceed the threshold, and in other embodiments the thresholds may be different. Accordingly, the thresholds that the vibration data is compared to may differ depending on the use case.

In some embodiments, the processing circuit 110 receives the vibration data in the form of a preprocessed electrical signal from the vibration sensor 120 as described above. In some embodiments, the processing circuit 110 performs the preprocessing steps. Afterwards, the processing circuit 110 evaluates the vibration data to determine characteristics of the detected vibrations. While other characteristics of the vibrations can be detected and analyzed, in some examples, the processing circuit 110 determines an amplitude and a frequency of the detected vibrations where the amplitude indicates a strength of the vibrations and the frequency indicates how often the vibrations occur within a specific time frame.

The processing circuit 110 compares the vibration data to predefined thresholds that can be indicative of an adequate operating condition for the medical imaging system 200 onboard the mobile medical imaging vehicle 300. These predefined thresholds can be set based on a design specification or operational standards for the medical imaging system 200 onboard the mobile medical imaging vehicle 300. For example, if the amplitude of the vibrations exceeds the threshold, indicating excessive vibration intensity, or if the frequency of the vibrations deviates from acceptable ranges, the processing circuit 110 can determine that the vibrations exceed the threshold for adequate operation of the medical imaging system 200.

At step 410, the processing circuit 110 of the sensor system 100 onboard the mobile medical imaging vehicle 300 provides an output. In some embodiments, the output is provided via the user interface 130. When the processing circuit 110 determines that the detected vibrations exceed a threshold, the processing circuit 110 can provide a range of outputs to the operator depending on the severity of the vibrations. In some embodiments, the processing circuit 110 provides a visual notification such as a popup notification on the display device 132 notifying the operator using text or images that the vibrations have exceeded the threshold and that the operator should relocate the mobile medical imaging vehicle 300 to a new, second location. In some embodiments, the visual notification comprises flashing lights or an animation. In some embodiments, the processing circuit 110 provides an audible notification over one or more speakers located onboard the mobile medical imaging vehicle 300. The audible notification can be a warning sound, an alarm, or spoken words. In some embodiments, the notification can include specific information about the nature of the threshold exceeded, such as the amplitude, frequency, and duration of the vibrations. The notification can also provide the threshold that was exceeded and how much the detected vibrations surpassed the threshold to enable the operator of the mobile medical imaging vehicle 300 to assess the situation and determine whether the mobile medical imaging vehicle 300 should be relocated.

At step 412, the mobile medical imaging vehicle 300 is relocated to a second location different from the first location. In some embodiments, the mobile medical imaging vehicle 300 is relocated based on the sensor system 100 determining that the received vibration data exceeds a threshold. The threshold may be indicative of a frequency or magnitude of the vibrations being great enough to affect medical images obtained by the medical imaging system 200 onboard the mobile medical imaging vehicle 300. Upon arriving at the second location, method 400 may be repeated such that the sensor system 100 onboard the mobile medical imaging vehicle 300 is once again used to determine whether the second location is suitable to perform the medical imaging process. The method 400 can be repeated as many times as necessary until a suitable location to perform the medical imaging process is identified. In some embodiments, for example in cases where the operator of the mobile medical imaging vehicle 300 does not prefer to move the mobile medical imaging vehicle 300 or the mobile medical imaging vehicle 300 is located at the first location for a long-term period of time, step 412 may be omitted and the mobile medical imaging vehicle 300 may remain at the first location and medical imaging procedures may be put on hold or rescheduled until a later time or date when the sensor system 100 determines that detected vibrations do not exceed the threshold that would affect medical images obtained by the medical imaging system 200 onboard the mobile medical imaging vehicle 300.

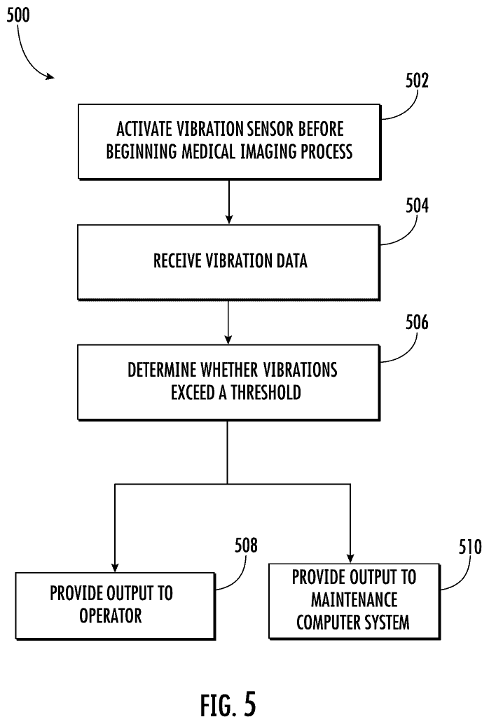

Referring to FIG. 5, a flow chart of a method 500 of operating a sensor system of a medical imaging system prior to performing a medical imaging process is shown according to an example embodiment. In some embodiments, the sensor system referred to by method 500 is the sensor system 100 described above with reference to FIG. 1, and the medical imaging system referred to by method 500 is the medical imaging system 200 described above with reference to FIG. 2. Accordingly, in some embodiments, the medical imaging system referred to by method 500 is the medical imaging system 200 onboard the mobile medical imaging vehicle 300 described above with reference to FIG. 3. In some embodiments, one or more steps of method 500 may be implemented as executable instructions stored in memory 114 such that the memory 114 is part of the medical imaging system 200 or that is onboard the mobile medical imaging vehicle 300.

At step 502, a vibration sensor is activated. The vibration sensor may be the same as the vibration sensor 120 described above with reference to the medical imaging system 200 of FIG. 2 or the mobile medical imaging vehicle 300 of FIG. 3. In some embodiments, the vibration sensor 120 is or includes a seismometer, a geophone, an accelerometer, or an infrasound sensor, among other sensor types.

In some embodiments, when a sensor 120 of the sensor system 100 is activated, the sensor 120 receives vibration data. In other words, the sensor 120 detects mechanical movement caused by vibrations in its environment. The mechanical movement is then converted into an electrical signal through a transducer of the sensor 120, which in some examples can be a piezoelectric element, a coil, or an accelerometer mass. In some embodiments, the sensor 120 is configured to preprocess the electrical signal before providing an output to the processing circuit 110, such as by conditioning the electrical signal by amplifying, filtering, or digitizing the electrical signal to clarify the signal and reduce noise. The sensor 120 is configured to provide the preprocessed signal to the processing circuit 110 as vibration data where it can be further analyzed or processed.

At step 504, the processing circuit 110 of the sensor system 100 of the medical imaging system 200 or that is onboard the mobile medical imaging vehicle 300 receives vibration data.

At step 506, the processing circuit 110 of the sensor system 100 of the medical imaging system 200 or that is onboard the mobile medical imaging vehicle 300 determines whether the received vibration data is indicative of vibrations exceeding a threshold. In determining whether the received vibration data is indicative of vibrations exceeding a threshold, the processing circuit 110 can compare one or more characteristics of the vibration data to a plurality of thresholds. For example, the thresholds can include at least one of an amplitude threshold defining a maximum allowable vibration level or a frequency threshold defining one or more frequencies or frequency ranges. In some embodiments, the one or more thresholds can include a duration or a pattern. For example, in some embodiments, short-term transient vibrations may not pose a significant risk to obtaining certain types of medical images but prolonged or repetitive vibrations may pose a greater risk to medical imaging equipment, and therefore prolonged or repetitive vibrations may exceed the threshold, and in other embodiments the thresholds may be different. Accordingly, the thresholds that the vibration data is compared to may differ depending on the use case.

In some embodiments, the processing circuit 110 determines whether the vibrations exceeding the threshold is indicative of a maintenance issue. For example, deviations in frequency or amplitude of vibrations detected when the medical imaging system 200 is running or operational, but before conducting a medical imaging process on a patient, (e.g., such as during an initiative or startup of the device) can be indicative of abnormal operational conditions or potential equipment malfunction. As such, the thresholds used to determine if the vibrations are indicative of a maintenance issue may differ from the thresholds used to determine if image quality would be compromised. For example, in determining whether a maintenance issue exists, the processing circuit 110 can evaluate how long the vibrations persist and whether they follow a recognizable pattern, such as continuous oscillations or random spikes, as prolonged or repetitive vibrations may be more likely to lead to wear, miscalibration, or damage of the medical equipment.

At step 508, the processing circuit 110 of the sensor system 100 of the medical imaging system 200 or that is onboard the mobile medical imaging vehicle 300 provides an output. In some embodiments, the output is provided via the user interface 130. When the processing circuit 110 determines that the detected vibrations exceed a threshold, the processing circuit 110 can provide a range of outputs to the operator depending on the severity of the vibrations. In some embodiments, the processing circuit 110 provides a visual notification such as a popup notification on the display device 132 notifying the operator using text or images that the vibrations have exceeded the threshold and that the operator should not operate the medical imaging equipment any longer or use the medical imaging equipment to obtain medical images of a patient as doing so may cause damage to the medical imaging equipment or result in medical images having poor image quality (e.g., blurry images or incomplete images). In some embodiments, the visual notification comprises flashing lights or an animation. In some embodiments, the processing circuit 110 provides an audible notification over one or more speakers. The audible notification can be a warning sound, an alarm, or spoken words. In some embodiments, the notification can include specific information about the nature of the threshold exceeded, such as the amplitude, frequency, and duration of the vibrations. The notification can also provide the threshold that was exceeded and how much the detected vibrations surpassed the threshold to enable the operator to assess the situation and determine whether to proceed with a medical imaging process.

At step 510, the processing circuit 110 of the sensor system 100 of the medical imaging system 200 or that is onboard the mobile medical imaging vehicle 300 provides an output to a maintenance computer system. The maintenance computer system (not shown) may be part of the sensor system 100 or may be an external or third-party computer system. For example, in an embodiment where the medical imaging system 200 is used in a traditional brick-and-mortar location such as a hospital or a clinic, the maintenance computer system can be located onsite at the brick-and-mortar location, or may be located at a location external to the brick-and-mortar location, such as at the location of a third-party, such as a at a location of a company that services medical imaging systems, or at a location a company that manufactured the medical imaging system 200. In another example, in an embodiment where the medical imaging system 200 is used onboard the mobile medical imaging vehicle 300, the maintenance computer system can be located onboard the mobile medical imaging vehicle 300, onsite at a brick-and-mortar location associated with the mobile medical imaging vehicle 300, or may be located at another external location, such as at the location of a third-party, such as a at a location of a company that services medical imaging systems, or at a location a company that manufactured the medical imaging system 200.

In some embodiments, the output provided to the maintenance computer system can provide an indication that the medical imaging system 200 requires maintenance based on the received vibration data. For example, if the sensor system determines at step 506 that the vibrations exceed a threshold, the sensor system 100 can determine if the vibrations are indicative of a maintenance issue as opposed to a non-maintenance issue. For example, a maintenance issue may include one or more of machine components being out of line, excessive wear of bearings, structural looseness, among other possible maintenance issues. In some embodiments, the sensor system 100 is configured to determine a likely maintenance issue based on one or more characteristics of the vibration data matching a known characteristic of a known potential maintenance issue within a threshold, and provide an indication of the likely maintenance issue to the maintenance computing system. In some embodiments, the sensor system 100 is configured to determine the vibration data exceeds the threshold at step 506, and provide an output to the maintenance computer system at step 510 accordingly. The output provided to the maintenance computer system can include an identification of the medical imaging system 200, a serial number of the medical imaging system 200, a model number of the medical imaging system 200, a location of the medical imaging system 200, a time and date when the vibration data was received at step 504, a date that the medical imaging system 200 last received maintenance, among other information.

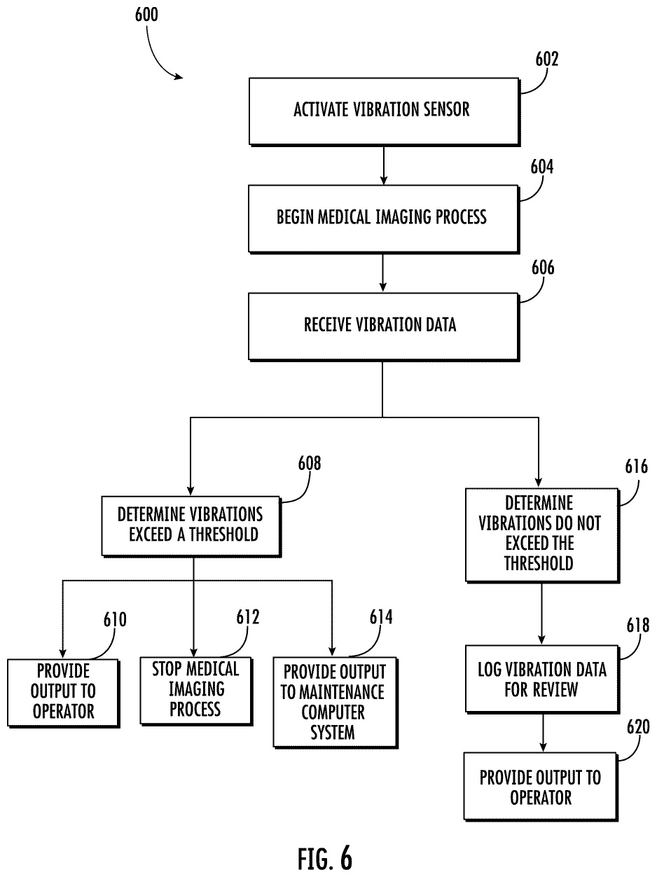

Referring to FIG. 6, a flow chart of a method 600 of operating a sensor system of the medical imaging system during a medical imaging process is shown according to an example embodiment. In some embodiments, the sensor system referred to by method 600 is the sensor system 100 described above with reference to FIG. 1, and the medical imaging system referred to by method 600 is the medical imaging system 200 described above with reference to FIG. 2. Accordingly, in some embodiments, the medical imaging system referred to by method 600 is the medical imaging system 200 onboard the mobile medical imaging vehicle 300 described above with reference to FIG. 3. In some embodiments, one or more steps of method 600 may be implemented as executable instructions stored in memory 114 such that the memory 114 is part of the medical imaging system 200 or that is onboard the mobile medical imaging vehicle 300.

At step 602, a vibration sensor is activated. The vibration sensor may be the same as the vibration sensor 120 described above with reference to the medical imaging system 200 of FIG. 2 or the mobile medical imaging vehicle 300 of FIG. 3. In some embodiments, the vibration sensor 120 is or includes a seismometer, a geophone, an accelerometer, or an infrasound sensor, among other sensor types. While step 602 is shown as the first step of method 600, it will be appreciated that activation of the vibration sensor 120 may occur after the medical imaging process begins at step 604, consistent with the description herein.

At step 604, the processing circuit 110 causes a medical imaging process to begin or otherwise be initiated. After preparing the patient 285, setting up the medical imaging system 200 for the correct protocol using the user interface 130, the medical imaging technician begins a medical imaging process by initiating the scan using the user interface 130.

At step 606, the processing circuit 110 of the sensor system 100 of the medical imaging system 200 or that is onboard the mobile medical imaging vehicle 300 receives vibration data. The vibration data is detected by the sensors 120 during the medical imaging process. In some embodiments, the sensor 120 detects mechanical movement caused by vibrations in its environment. The mechanical movement is then converted into an electrical signal through a transducer of the sensor 120, which in some examples can be a piezoelectric element, a coil, or an accelerometer mass. In some embodiments, the sensor 120 is configured to preprocess the electrical signal before providing an output to the processing circuit 110, such as by conditioning the electrical signal by amplifying, filtering, or digitizing the electrical signal to clarify the signal and reduce noise. The sensor 120 is configured to provide the preprocessed signal to the processing circuit 110 as vibration data where it can be further analyzed or processed.

At step 608, the sensor system 100 of the medical imaging system 200 or that is onboard the mobile medical imaging vehicle 300 determines that the vibrations exceed a threshold. In determining that the received vibration data is indicative of vibrations exceeding a threshold, the processing circuit 110 can compare one or more characteristics of the vibration data to a plurality of thresholds. For example, the thresholds can include at least one of an amplitude threshold defining a maximum allowable vibration level or a frequency threshold defining one or more frequencies or frequency ranges. In some embodiments, the one or more thresholds can include a duration or a pattern. For example, in some embodiments, short-term transient vibrations may not pose a significant risk to obtaining certain types of medical images but prolonged or repetitive vibrations may pose a greater risk to medical imaging equipment, and therefore prolonged or repetitive vibrations may exceed the threshold, and in other embodiments the thresholds may be different. Accordingly, the thresholds that the vibration data is compared to may differ depending on the use case.

At step 610, the processing circuit 110 of the sensor system 100 of the medical imaging system 200 or that is onboard the mobile medical imaging vehicle 300 provides an output to the technician. In some embodiments, the output is provided via the user interface 130. When the processing circuit 110 determines that the detected vibrations exceed a threshold, the processing circuit 110 can provide a range of outputs to the operator depending on the severity of the vibrations. In some embodiments, the processing circuit 110 provides a visual notification such as a popup notification on the display device 132 notifying the operator using text or images that the vibrations have exceeded the threshold and that the operator should not operate the medical imaging equipment any longer or use the medical imaging equipment to obtain medical images of a patient as doing so may cause damage to the medical imaging equipment or result in medical images having poor image quality (e.g., blurry images or incomplete images). In some embodiments, the visual notification comprises flashing lights or an animation. In some embodiments, the processing circuit 110 provides an audible notification over one or more speakers. The audible notification can be a warning sound, an alarm, or spoken words. In some embodiments, the notification can include specific information about the nature of the threshold exceeded, such as the amplitude, frequency, and duration of the vibrations. The notification can also provide the threshold that was exceeded and how much the detected vibrations surpassed the threshold to enable the operator to assess the situation and determine whether to proceed with a medical imaging process.

At step 612, the processing circuit 110 of the sensor system 100 of the medical imaging system 200 or that is onboard the mobile medical imaging vehicle 300 causes the medical imaging system 200 to stop the medical imaging process. The processing circuit 110 may automatically cause the medical imaging process to stop. In some embodiments, the processing circuit 110 causes the medical imaging process to stop based on a severity of the vibrations. For example, at step 608, the processing circuit 110 can determine that the vibrations not only exceed the threshold but also a higher level threshold indicating that continued operation of the medical imaging system 200 may cause the medical imaging system 200 to become damaged or that the patient 285 may become harmed if the medical imaging process continues. In such embodiments, the processing circuit 110 acts as a safeguard and automatically stops the medical imaging process.

At step 614, the processing circuit 110 of the sensor system 100 of the medical imaging system 200 or that is onboard the mobile medical imaging vehicle 300 provides an output to a maintenance computer system. The processing circuit 110 may provide an output to a maintenance computer system similar to the processes described above with reference to step 510.

At step 616, the sensor system 100 of the medical imaging system 200 or that is onboard the mobile medical imaging vehicle 300 determines that the vibrations do not exceed the threshold. In this step, the processing circuit 110 can perform the same functions with respect to step 608, but determine that the vibrations do not exceed the threshold.

At step 618, the processing circuit 110 of the sensor system 100 of the medical imaging system 200 or that is onboard the mobile medical imaging vehicle 300 logs the vibration data in memory for review. In some embodiments, the processing circuit 110 logs the vibration data in memory 114 or in another memory. Even when the processing circuit 110 determines that vibration data does not exceed a threshold, the data may be logged for a predetermined amount of time to enable technicians or maintenance personnel to track patterns, identify recurring issues, and assess equipment performance over extended periods, even if the vibrations do not indicate any present issues with the medical imaging system 200.

At step 620, the processing circuit 110 of the sensor system 100 of the medical imaging system 200 or that is onboard the mobile medical imaging vehicle 300 provides an output to the operator. The processing circuit 110 may provide an output to the operator similar to the processes described above with reference to step 610.

Referring to FIG. 7, an illustration of a work vehicle 700 comprising the sensor system 100 of FIG. 1 is shown according to an example embodiment. The work vehicle 700 includes a plurality of outriggers 702 and a lifting apparatus 704. The work vehicle 700 includes the sensor system 100 of FIG. 1, including the processing circuit 110, the user interface 130, and the sensors 120, which are shown to be coupled with the outriggers 702. In some embodiments, the work vehicle 700 is a crane, though it will be appreciated that the work vehicle 700 can be any type of work vehicle. For example, the work vehicle 700 can be a bucket loader, a backhoe, a loader, a fire engine, or other construction vehicle. In some embodiments, the work vehicle 700 is a mobile medical imaging vehicle, such as the mobile medical imaging vehicle 300 described above with reference to FIG. 3.

The work vehicle 700 can be configured to lift heavy objects, such as medical equipment, including the medical imaging system 200, or components thereof. For example, in one embodiment, the work vehicle 700 is capable of lifting heavy magnets for MR imaging systems into medical buildings where MR imaging systems are located so that the magnets can be installed in the MR imaging systems.

The work vehicle 700 can include a base or chassis that provides stability during operation of the work vehicle 700. The base of the work vehicle 700 can be mounted on a truck or crawler tracks, allowing the work vehicle 700 to be driven by an operator to move the work vehicle 700 about a worksite. In some embodiments, the work vehicle 700 is typically transported via a trailer to a worksite and then unloaded before being driven about the worksite and positioned to lift one or more objects.

The plurality of outriggers 702 extend from the base of the work vehicle 700. The outriggers 702 are stabilizing arms that support the work vehicle 700 to ensure that the work vehicle 700 remains steady when lifting heavy loads. The outriggers 702 also distribute the weight and prevent the work vehicle 700 from tipping over when lifting loads, or when lifting loads on uneven terrain. For example, when the outriggers 702 are deployed, the outriggers 702 transfer the weight from the wheels or tracks of the work vehicle 700 to the ground, effectively increasing the footprint of the work vehicle 700 and ensuring better balance, especially when the work vehicle 700 handles loads that are off-center or lifted to significant heights.

The outriggers 702 can be selectively extended and retracted by providing a user input on the user interface 130 onboard the work vehicle 700. In some embodiments, the outriggers 702 are extended and retracted by a hydraulic system, enabling the outriggers 702 to be quickly and precisely positioned.

The outriggers 702 can take many forms and typically include a beam that extends from the base of the work vehicle 700 and the beam is coupled with a metal plate that interfaces the ground. In some embodiments, the outriggers 702 can be individually adjusted to account for uneven terrain, with each outrigger 702 extending to a different length or exerting a different amount of pressure to maintain a level base for the work vehicle 700. Example embodiments of the outriggers 702 are illustrated in FIG. 8A and FIG. 8B.

In some embodiments, the outriggers 702 include built-in safety features and monitoring systems to ensure that the outriggers 702 are used effectively. In some embodiments, each outrigger 702 includes a dedicated sensor 120. In some embodiments, the sensors 120 can be used to provide real-time feedback to an operator of the work vehicle 700 regarding the placement of the outriggers 702. In some embodiments, the sensor system 100 onboard the work vehicle 700 can detect the pressure being exerted on each outrigger 702 to ensure each outrigger 702 is being placed on stable ground. In some embodiments, the sensor system 100 onboard the work vehicle 700 is configured to detect underground voids beneath each outrigger 702 before each outrigger 702 fully assumes the weight of the work vehicle 700 to ensure the outrigger 702 does not collapse into the underground void when supporting the work vehicle 700 or when greater loads are placed on the outrigger 702, such as when the work vehicle 700 lifts heavy objects. For example, the sensor system 100 can be configured to detect any kind of voids that may be hazardous to the operation of the work vehicle 700, including but not limited to man holes, sewers, underground utility areas, or caves. As such, the sensors 120 on the outriggers 702 can be any type of sensor described above with reference to FIG. 1.

The lifting apparatus 704 can include any type of lifting equipment configured to lift heavy equipment, such as MRI magnets. For example, the lifting apparatus 704 can include a boom. In some embodiments, the boom can be telescopic and therefore can be extended or retracted based on the specific parameters of a job to enable the work vehicle 700 to access various heights and distances. For example, in some embodiments, the boom can be extended to enable the work vehicle 700 to lift equipment into a room in a hospital or clinic, where the room can be on a ground floor of the building or on a higher floor of the building.

The lifting apparatus 704 can also include a hoist and winch system. The hoist and winch system is configured to lift and lower the equipment by controlling movement of a cable. For example, the hoist can be attached to the end of the boom and use a system of pulleys and motors to provide the mechanical force necessary to lift the equipment. The winch is powered by a motor and controls the tension and movement of the cable.

The user interface 130 can include any type of control elements configured to enable an operator of the work vehicle 700 to control the work vehicle 700 and the sensor system 100 onboard. The user interface 130 can include any feature or functionality described above with reference to FIG. 1.

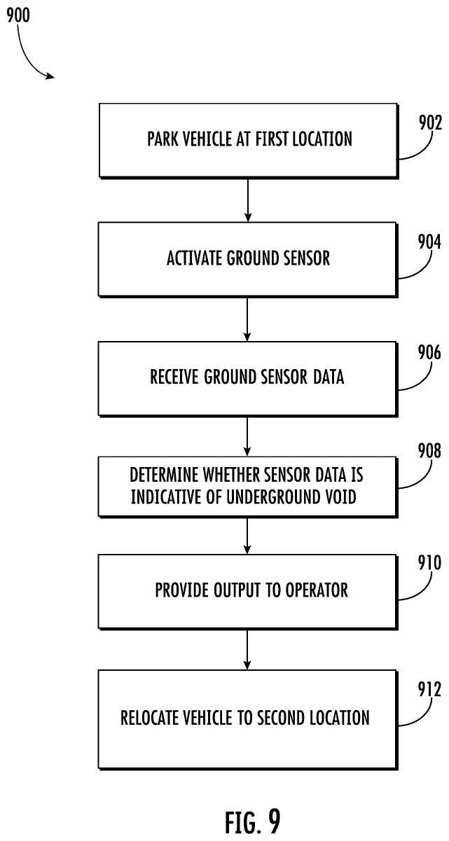

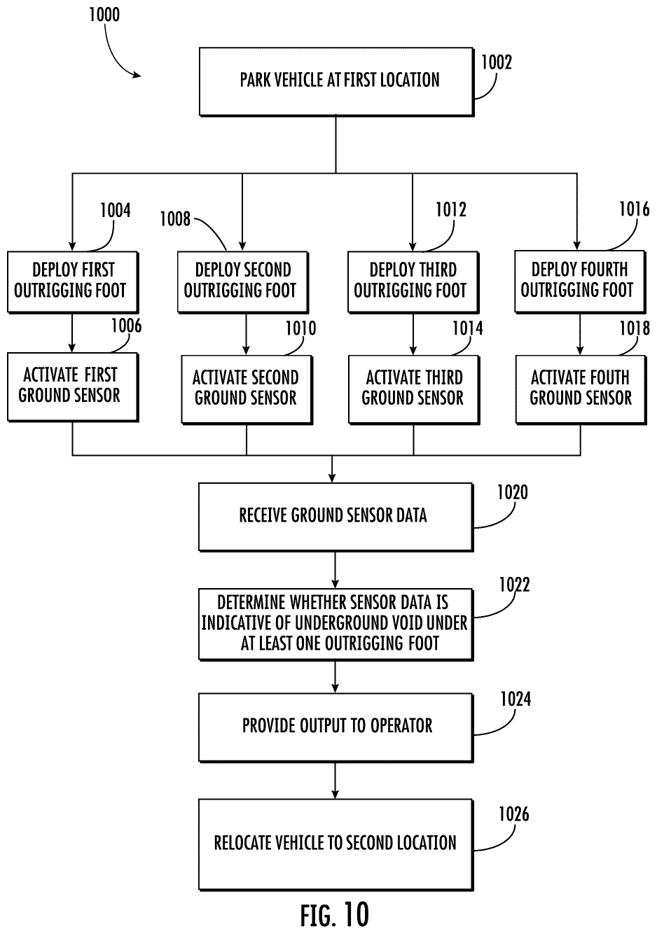

The processing circuit is configured to control the operations of the outriggers 702 and the sensors 120 and provide outputs on the user interface 130 based on user inputs received via the user interface 130. For example, based on a user input, the processing circuit 110 can control the deployment of the outriggers 702, activate the sensors 120 to scan for underground voids, and provide outputs based on the scans on the user interface 130. The processing circuit 110 can carry out all processes described herein with respect to FIG. 1, FIG. 9, and FIG. 10.

Referring to FIG. 8A and FIG. 8B, illustrations of outriggers 702 comprising a sensor 120 of the sensor system 100 of FIG. 1 are shown according to example embodiments. As shown in FIG. 8A, a horizontal outrigger 702 is shown. The horizontal outrigger includes a first beam extending from the base of the work vehicle 700, a second beam coupled to and perpendicular to the first beam, and a plate configured to interface with the ground. The first beam is configured to hydraulically extend from the work vehicle 700, and then the second beam is configured to be hydraulically lowered with respect to the first beam to cause the plate to contact the ground and to support the work vehicle 700. As shown in FIG. 8B, an angled outrigger 702 is shown. The angled outrigger 702 includes a telescoping beam that extends from the base of the work vehicle 700 at an angle to cause the plate to contact the ground to support the work vehicle 700.

Referring to FIG. 9, a flow chart of a method 900 of operating the sensor system of a work vehicle is shown according to an example embodiment. In some embodiments, the sensor system referred to by method 900 is the sensor system 100 described above with reference to FIG. 1, and the work vehicle referred to by method 900 is the work vehicle 700 described above with reference to FIG. 7. In some embodiments, one or more steps of method 900 may be implemented as executable instructions stored in memory onboard the work vehicle, such as memory 114.

At step 902, the work vehicle 700 is parked at a first location. Typically, an operator of the work vehicle 700 will unload the work vehicle 700 from a trailer at a work site, and then drive the work vehicle 700 to a first location at the work site in which the work vehicle 700 is needed to perform a duty, such as lifting a component of a medical imaging device (e.g., a component of medical imaging system 200) into a building for installation. In some embodiments, if the work vehicle 700 has self-driving capabilities, the work vehicle 700 can drive to the location without the assistance of a human operator, or with some assistance from the human operator depending on the level of the self-driving capabilities of the work vehicle 700. In some embodiments, the work vehicle 700 is already located at the first location.

At step 904, a ground sensor onboard the work vehicle 700 is activated. The ground sensor may be the same as the sensors 120 described above with reference to the sensor system 100 described in FIG. 1. In some embodiments, the ground sensor is a ground-penetrating radar sensor, a microgravity sensor, or a seismic sensor such as a vibroseis device or a hammer and plate device. The ground sensor is configured to sense at least one of seismic activity or underground voids, and to generate ground sensor data.

In some embodiments, when a sensor 120 of the sensor system 100 is activated, the sensor 120 receives ground sensor data. In other words, the sensor 120 detects voids based on received seismic waves or based on received radar signals. In some embodiments, the received waves or signals are converted into an electrical signal. In some embodiments, the sensor 120 is configured to preprocess the electrical signal before providing an output to the processing circuit 110, such as by conditioning the electrical signal by amplifying, filtering, or digitizing the electrical signal to clarify the signal and reduce noise. The sensor 120 is configured to provide the preprocessed signal to the processing circuit 110 as ground sensor data where it can be further analyzed or processed.

At step 906, the sensor system 100 onboard the work vehicle 700 receives ground sensor data from one or more of the sensors 120.

At step 908, the processing circuit 110 of the sensor system 100 determines whether the sensor data is indicative of an underground void being beneath the work vehicle 700. In some embodiments, the processing circuit 110 can compare the sensor data against known patterns of stable ground and voids, using pre-programmed models that define how normal versus abnormal subsurface conditions should behave. The processing circuit 110 can then detect anomalies in the processed data that may signal the presence of an underground void. The processing circuit 110 can evaluate the anomalies against established thresholds that indicate whether the ground is stable or compromised. For example, the processing circuit 110 can detect that the sensor data is indicative of a significant drop in soil density. In another example, the processing circuit 110 can detect that abnormal subsurface vibrations indicate the presence of a void. The processing circuit 110 can compare such determinations to predetermined thresholds as part of determining the likelihood of an underground void being present under the work vehicle 700.

At step 910, the processing circuit 110 of the sensor system 100 onboard the work vehicle 700 provides an output. In some embodiments, the output is provided via the user interface 130. When the processing circuit 110 determines that the sensor data is indicative of an underground void under the work vehicle 700, the processing circuit 110 can provide a range of outputs to the operator depending on a characteristic of the detected underground void. In some embodiments, the processing circuit 110 provides a visual notification such as a popup notification on the display device 132 notifying the operator using text or images that the underground void poses a danger to the safe operation of the work vehicle 700 and that the operator should relocate the work vehicle 700 to a new, second location. In some embodiments, the notification can be to merely reposition the work vehicle 700 in the same general location such that the outriggers 702 are in new positions and will interface with the ground in new positions when the work vehicle 700 is repositioned. In some embodiments, the visual notification comprises flashing lights or an animation. In some embodiments, the processing circuit 110 provides an audible notification over one or more speakers located onboard the work vehicle 700. The audible notification can be a warning sound, an alarm, or spoken words. In some embodiments, the notification can include specific information about the nature of the underground void, such as a size of the void, a specific location of the void, a starting depth of the void, and an ending depth of the void. For example, the processing circuit 110 may be able to distinguish between a limited void (e.g., a single sewer tunnel) that may require the work vehicle 700 to merely be repositioned and a more expansive void (e.g., a large underground cave) that may require the work vehicle 700 to be relocated.

At step 912, the work vehicle 700 is relocated to a second location different from the first location, or the work vehicle 700 is repositioned. In some embodiments, the work vehicle 700 is relocated based on the sensor system 100 determining that the received ground sensor data indicates that an underground void is present beneath the work vehicle 700. Upon repositioning the work vehicle 700 or upon the work vehicle 700 arriving at the second location, method 900 may be repeated such that the sensor system 100 onboard the work vehicle 700 is once again used to determine whether the work vehicle 700 can perform a task such as lifting a heavy object. The method 900 can be repeated as many times as necessary until a suitable location and position to perform the task is identified.

Referring to FIG. 10, another flow chart of a method 1000 of operating the sensor system of a work vehicle is shown according to an example embodiment. In some embodiments, the sensor system referred to by method 1000 is the sensor system 100 described above with reference to FIG. 1, and the work vehicle referred to by method 1000 is the work vehicle 700 described above with reference to FIG. 7. In some embodiments, one or more steps of method 1000 may be implemented as executable instructions stored in memory onboard the work vehicle 700, such as memory 114.

At step 1002, the work vehicle 700 is parked at a first location. This step may be the same as or substantially similar to step 902, described above with reference to FIG. 9.

At step 1004, a first outrigger 702 of the work vehicle 700 is deployed. The first outrigger 702 can be deployed based on a user input received on the user interface 130.

At step 1006, a first ground sensor 120 is activated. The first ground sensor 120 is located on the first outrigger 702. The first ground sensor 120 may be the same as the sensors 120 described above with reference to FIG. 1 and FIG. 7.

At step 1008 and step 1010, a second outrigger 702 of the work vehicle 700 is deployed, and a second ground sensor 120 is activated, respectively. Steps 1008 and 1010 may be the same as or substantially similar to steps 1004 and 1006, respectively, described above.

At step 1012 and step 1014, a third outrigger 702 of the work vehicle 700 is deployed, and a third ground sensor 120 is activated, respectively. Steps 1012 and 1014 may be the same as or substantially similar to steps 1004 and 1006, respectively, described above.

At step 1016 and step 1018, a fourth outrigger 702 of the work vehicle 700 is deployed, and a fourth ground sensor 120 is activated, respectively. Steps 1016 and 1018 may be the same as or substantially similar to steps 1004 and 1006, respectively, described above.

At step 1020, the processing circuit 110 of the sensor system 100 receives ground sensor data. In some embodiments, the processing circuit 110 receives ground sensor data from each of the sensors 120 associated with each outrigger 702 at the same time or near-simultaneous. In some embodiments, the processing circuit 110 receives ground sensor data from each sensor 120 sequentially. For example, the processing circuit 110 can receive ground sensor data at step 1020 immediately after completion of step 1006 when the first ground sensor is activated. In another example, the processing circuit 110 can receive ground sensor data at step 1020 immediately after completion of step 1010 when the second ground sensor is activated, and so on. In other words, beginning step 1020 does not depend on first completing each of steps 1006, 1010, 1014, and 1018. For example, in some embodiments, the processing circuit receives ground sensor data at step 1020 after the first outrigger 702 is deployed and the first sensor 120 on the first outrigger 702 is activated, the system determines that the sensor data is indicative of an underground void at step 1022, an output is provided to the operator at step 1024, and the work vehicle 700 is relocated or repositioned at step 1026 before the second outrigger 702, third outrigger 702, or fourth outrigger 702 are deployed at step 1008, step 1012, or step 1016, respectively.

At step 1022, the processing circuit 110 of the sensor system 100 determines whether the sensor data is indicative of an underground void being beneath the work vehicle 700. This step may be the same as or substantially similar to step 908, described above with reference to FIG. 9. However, given that the processing circuit 110 can receive sensor data sequentially from each sensor 120, the processing circuit 110 at step 1022 can also sequentially determine whether sensor data received from a particular sensor 120 is indicative of an underground void before determining whether sensor data received from another sensor 120 is indicative of an underground void. For example, the processing circuit 110 can determine that sensor data from a first sensor 120 on a first outrigger 702 is indicative of an underground void being underneath the first outrigger 702 before determining whether sensor data from a second sensor 120 on a second outrigger 702 is indicative of an underground void.

At step 1024, the processing circuit 110 of the sensor system 100 provides an output. This step may be the same as or substantially similar to step 910, described above with reference to FIG. 9. In some embodiments, the output specifies the specific outrigging foot that the underground void was detected beneath. In some embodiments, a narrower void, such as a sewer tunnel, may require repositioning of the work vehicle 700 or disablement or non-use of one outrigging foot as opposed to relocating the work vehicle 700. However, a larger void, such as an underground cave system, may require the work vehicle 700 to be relocated to a second location where the larger void will not affect operation of the work vehicle 700.

At step 1026, the work vehicle 700 is relocated to a second location different from the first location, or the work vehicle 700 is repositioned. This step may be the same as or substantially similar to step 912, described above with reference to FIG. 9.

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As utilized herein, terms of degree such as “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to any precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that terms such as “exemplary,” “example,” and similar terms, as used herein to describe various embodiments, are intended to indicate that such embodiments are possible examples, representations, or illustrations of possible embodiments, and such terms are not intended to connote that such embodiments are necessarily extraordinary or superlative examples.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any element on its own or any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the drawings. It should be noted that the orientation of various elements may differ according to other exemplary embodiments, and that such variations are intended to be encompassed by the present disclosure.

As used herein, terms such as “engine” or “circuit” may include hardware and machine-readable media storing instructions thereon for configuring the hardware to execute the functions described herein. The engine or circuit may be embodied as one or more circuitry components including, but not limited to, processing circuitry, network interfaces, peripheral devices, input devices, output devices, sensors, etc. In some embodiments, the engine or circuit may take the form of one or more analog circuits, electronic circuits (e.g., integrated circuits (IC), discrete circuits, system on a chip (SOCs) circuits, etc.), telecommunication circuits, hybrid circuits, and any other type of circuit. In this regard, the engine or circuit may include any type of component for accomplishing or facilitating achievement of the operations described herein. For example, an engine or circuit as described herein may include one or more transistors, logic gates (e.g., NAND, AND, NOR, OR, XOR, NOT, XNOR, etc.), resistors, multiplexers, registers, capacitors, inductors, diodes, wiring, and so on).

An engine or circuit may be embodied as one or more processing circuits comprising one or more processors communicatively coupled to one or more memory or memory devices. In this regard, the one or more processors may execute instructions stored in the memory or may execute instructions otherwise accessible to the one or more processors. The one or more processors may be constructed in a manner sufficient to perform at least the operations described herein. In some embodiments, the one or more processors may be shared by multiple engines or circuits (e.g., engine A and engine B, or circuit A and circuit B, may comprise or otherwise share the same processor which, in some example embodiments, may execute instructions stored, or otherwise accessed, via different areas of memory).

Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be provided as one or more suitable processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively or additionally, the one or more processors may be internal and/or local to the apparatus. In this regard, a given engine or circuit or components thereof may be disposed locally (e.g., as part of a local server, a local computing system, etc.) or remotely (e.g., as part of a remote server such as a cloud based server). To that end, engines or circuits as described herein may include components that are distributed across one or more locations.

An example system for providing the overall system or portions of the embodiments described herein might include one or more computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some embodiments, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components, etc.), in accordance with the example embodiments described herein.

Although the drawings may show and the description may describe a specific order and composition of method steps, the order of such steps may differ from what is depicted and described. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions, and arrangement of the embodiments without departing from the scope of the present disclosure as expressed in the appended claims.

What is claimed is:

1. A system comprising:
a vibration sensor configured to sense vibrations and to generate vibration data based on the sensed vibrations;
a medical imaging device coupled with the vibration sensor and comprising a display device, the medical imaging device configured to obtain medical images of a patient; and
a processing circuit configured to:
receive the vibration data from the vibration sensor; and
provide an output on the display device based on the vibration data.

2. The system of claim 1, wherein the vibration sensor is a seismic sensor, and the seismic sensor comprises at least one of a seismometer, geophone, an accelerometer, or an infrasound sensor.

3. The system of claim 1, wherein the medical imaging device is a magnetic resonance (MR) imaging device.

4. The system of claim 1, wherein the vibration sensor is configured to generate vibration data prior to the medical imaging device obtaining a medical image of the patient, and the processing circuit is configured to provide the output prior to the medical imaging device obtaining the medical image of the patient.

5. The system of claim 4, wherein the output provides an indication of whether any vibrations were sensed and whether the medical imaging device can be operated to obtain a medical image without comprising the integrity of the medical image.

6. The system of claim 1, wherein the vibration sensor is configured to generate vibration data during a medical imaging process, and the processing circuit is configured to provide the output at least one of during the medical imaging process or after the medical imaging process.

7. The system of claim 6, wherein the output comprises an indication of whether any vibrations were sensed during the medical imaging process and whether the integrity of any obtained medical images is compromised.

8. The system of claim 7, wherein the output identifies a specific medical image of a plurality of the obtained medical images that is comprised based on the vibration data.

9. The system of claim 6, wherein the output causes the medical imaging device to stop the medical imaging process based on the vibration data.

10. The system of claim 1, wherein the vibration data is indicative of a component of the medical imaging device requiring maintenance, and wherein the output is a maintenance alert.

11. The system of claim 1, further comprising a vehicle, wherein the vibration sensor, the medical imaging device, and the processing circuit are located on the vehicle.

12. A system comprising:
a ground sensor system configured to sense at least one of seismic activity or underground voids, and to generate ground sensor data based on the sensed seismic activity;
a work vehicle comprising a seismic sensor and a display device, the work vehicle configured to lift equipment;
a processing circuit on the work vehicle and configured to:
receive the ground sensor data from the ground sensor system; and
provide an output on the display device based on the ground sensor data.

13. The system of claim 12, wherein the work vehicle comprises a plurality of outriggers, the ground sensor system comprises a plurality of ground sensors, and wherein each outrigger comprises a ground sensor.

14. The system of claim 13, wherein the processing circuit is configured to determine, based on the ground sensor data, whether any of the outriggers are positioned over an underground void based on the ground sensor data.

15. The system of claim 12, wherein the ground sensor is a seismic sensor, and the seismic sensor comprises at least one of a vibroseis device or a hammer and plate device.

16. The system of claim 15, wherein the seismic sensor is configured to generate seismic waves to penetrate the ground in proximity to the work vehicle.

17. A method comprising:
generating, by a vibration sensor configured to generate vibration data based on sensed vibrations, vibration data;
receiving, by a processing circuit of a medical imaging device configured to obtain medical images of a patient and comprising the vibration sensor and a display device, the vibration data from the vibration sensor; and
providing, by the processing circuit via the display device, an output based on the vibration data.

18. The method of claim 17, wherein the vibration sensor is a seismic sensor, the seismic sensor comprises at least one of a seismometer, geophone, an accelerometer, or an infrasound sensor, and the medical imaging device is at least one of a magnetic resonance (MR) imaging device, a computed tomography (CT) imaging device, a positron emission tomography (PET) imaging device, a single-photon emission computed tomography (SPECT) imaging device, or an X-ray imaging device.

19. The method of claim 17, further comprising:
generating, by the vibration sensor, the vibration data prior to the medical imaging device obtaining a medical image of the patient; and
providing, by the processing circuit, the output prior to the medical imaging device obtaining the medical image of the patient;
wherein the output provides an indication of whether any vibrations were sensed and whether the medical imaging device can be operated to obtain a medical image without comprising the integrity of the medical image.

20. The method of claim 17,
generating, by the vibration sensor, the vibration data during a medical imaging process; and
providing, by the processing circuit, the output at least one of during the medical imaging process or after the medical imaging process;
wherein the output comprises an indication of whether any vibrations were sensed during the medical imaging process and whether the integrity of any obtained medical images is compromised.
